(19) United States
(12) Patent Application Publication
(10) Pub. No.: US 2012/0123178 A1
(43) Pub. Date: May 17, 2012

(54) UZM-35HS ALUMINOSILICATE ZEOLITE, METHOD OF PREPARATION AND PROCESSES USING UZM-35HS

(75) Inventors: Jaime G Moscoso, Mount Prospect, IL (US); Deng-Yang Jan, Elk Grove Village, IL (US)

(73) Assignee: UOP LLC, Des Plaines, IL (US)

(21) Appl. No.: 13/353,742

(22) Filed: Jan. 19, 2012

Related U.S. Application Data
(63) Continuation-in-part of application No. 13/079,399, filed on Apr. 4, 2011.
Application No. 13/079,399 is a continuation of application No. 12/241,302, filed on Sep. 30, 2008, now Pat. No. 7,922,997.

(51) Int. Cl. C07C 2/12; C01B 39/46; C07C 2/00; C07C 7/148; C07C 5/22

(52) U.S. Cl. 585/533; 585/852; 585/899; 423/718

(57) ABSTRACT

A new family of crystalline aluminosilicate zeolites has been synthesized. These zeolites are represented by the empirical formula.

M1ₐⁿ⁺Al₍₁₋ₓ₎EₓSi_{y′}O_{z″}

where M1 is at least one exchangeable cation selected from the group consisting of alkali, alkaline earth metals, rare earth metals, ammonium ion, hydrogen ion and mixtures thereof, “a” is the mole ratio of M1 to (Al+E), “n” is the weighted average valence of M1, E is an element selected from the group consisting of gallium, iron, boron, and mixtures thereof, “x” is the mole fraction of E, y′ is the mole ratio of Si to (Al+E), and z″ is the mole ratio of O to (Al+E). These zeolites are similar to MCM-68 but are characterized by unique x-ray diffraction patterns and compositions and have catalytic properties for carrying out various hydrocarbon conversion processes.

### CROSS-REFERENCE TO RELATED APPLICATION

This application is a Continuation-In-Part of copending U.S. application Ser. No. 13/079,399 filed Apr. 4, 2011 which in turn is a Continuation of U.S. application Ser. No. 12/241,302 filed Sep. 30, 2008, now U.S. Pat. No. 7,922,997, the contents of which are hereby incorporated by reference in their entirety.

### FIELD OF THE INVENTION

This invention relates to a new family of aluminosilicate zeolites designated UZM-35HS. They are represented by the empirical formula of:

M1ₐⁿ⁺Al₍₁₋ₓ₎EₓSi_{y′}O_{z″}

where M1 is at least one exchangeable cation selected from the group consisting of alkali, alkaline earth metals, rare earth metals, ammonium ion, hydrogen ion and mixtures thereof, “a” is the mole ratio of M1 to (Al+E), “n” is the weighted average valence of M1, E is an element selected from the group consisting of gallium, iron, boron, and mixtures thereof, “x” is the mole fraction of E, y′ is the mole ratio of Si to (Al+E), and z″ is the mole ratio of O to (Al+E).

### BACKGROUND OF THE INVENTION

Zeolites are crystalline aluminosilicate compositions which are microporous and which are formed from corner sharing AlO₂ and SiO₂ tetrahedra. Numerous zeolites, both naturally occurring and synthetically prepared are used in various industrial processes. Synthetic zeolites are prepared via hydrothermal synthesis employing suitable sources of Si, Al and structure directing agents such as alkali metals, alkaline earth metals, amines, or organoammonium cations. The structure directing agents reside in the pores of the zeolite and are largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure. Zeolites can be used as catalysts for hydrocarbon conversion reactions, which can take place on outside surfaces as well as on internal surfaces within the pore.

One particular zeolite, designated MCM-68 was disclosed by Calabro et al. in 1999 (U.S. Pat. No. 6,049,018). This patent describes the synthesis of MCM-68 from dication directing agents, N,N,N′,N′-tetraalkylbicyclo[2.2.2.]oct-7-ene-2,3:5,6-dipyrrolidinium dication, and N,N,N′,N′-tetraalkylbicyclo[2.2.2.]octane-2,3:5,6-dipyrrolidinium dication. The MCM-68 was found to have at least one channel system in which each channel is defined by a 12-membered ring of tetrahedrally coordinated atoms and at least two further independent channel systems in which each channel is defined by a 10-membered ring of tetrahedrally coordinated atoms wherein the number of unique 10-membered ring channels is twice the number of 12-membered ring channels.

Applicants have successfully prepared a new family of materials designated UZM-35, as synthesized, and UZM-HS as modified. The topology of the materials is similar to that observed for MCM-68. The materials are prepared via the use of a simple commercially available structure directing agents, such as dimethyldipropylammonium hydroxide, in concert with small amounts of K⁺ and Na⁺ together using the Charge Density Mismatch Approach to zeolite synthesis (U.S. Pat. No. 7,578,993).

### SUMMARY OF THE INVENTION

As stated, the present invention relates to a new aluminosilicate zeolite designated UZM-35HS. As synthesized, UZM-35 is a microporous crystalline zeolite having a three-dimensional framework of at least AlO₂ and SiO₂ tetrahedral units and an empirical composition in the as synthesized and anhydrous basis expressed by an empirical formula of:

Mₘⁿ⁺R⁺ᵣAl₁₋ₓEₓSi_{y}O_{z}

where M represents a combination of potassium and sodium exchangeable cations, “m” is the mole ratio of M to (Al+E) and varies from about 0.05 to about 3, R is a singly charged organoammonium cation selected from the group consisting of dimethyldipropylammonium (DMDPA⁺), choline, ethyltrimethylammonium (ETMA⁺), diethyldimethylammonium (DEDMA⁺), trimethylpropylammonium, trimethylbutylammonium, dimethyldiethanolammonium, tetraethylammonium (TEA⁺), tetrapropylammonium (TPA⁺), methyltripropylammonium, and mixtures thereof, “r” is the mole ratio of R to (Al+E) and has a value of about 0.25 to about 2.0, E is an element selected from the group consisting of gallium, iron, boron and mixtures thereof, “x” is the mole fraction of E and has a value from 0 to about 1.0, “y” is the mole ratio of Si to (Al+E) and varies from greater than 2 to about 12 and “z” is the mole ratio of O to (Al+E) and has a value determined by the equation:

z=(m+r+3+4·y)/2

and is characterized in that it has the x-ray diffraction pattern having at least the d-spacings and intensities set forth in Table A

TABLE A

| 2θ | d (Å) | I/Io % |
|---|---|---|
| 6.45-6.8 | 13.7-13 | m |
| 6.75-7.13 | 13.1-12.4 | m-vs |
| 7.86-8.26 | 11.25-10.7 | m |
| 8.64-9.04 | 10.23-9.78 | m |
| 9.51-10.09 | 9.3-8.77 | m-vs |
| 10.62-11.23 | 8.33-7.88 | w-m |
| 13.4-14.22 | 6.61-6.23 | w-m |
| 14.76-15.55 | 6-5.7 | w |
| 17.63-18.37 | 5.03-4.83 | w |
| 19.17-19.91 | 4.63-4.46 | w-m |
| 19.64-20.56 | 4.52-4.32 | m |
| 20.18-21.05 | 4.4-4.22 | w-m |
| 20.7-21.57 | 4.29-4.12 | w-m |
| 21.36-22.28 | 4.16-3.99 | vs |
| 22.17-23.6 | 4.01-3.77 | m-s |
| 24.12-25.23 | 3.69-3.53 | w |
| 25.6-26.94 | 3.48-3.31 | m |
| 26.37-27.79 | 3.38-3.21 | m |
| 27.02-28.42 | 3.3-3.14 | m |
| 27.53-28.89 | 3.24-3.09 | m |
| 28.7-30.09 | 3.11-2.97 | m |
| 29.18-30.72 | 3.06-2.91 | w-m |
| 30.19-31.73 | 2.96-2.82 | m |
| 30.83-32.2 | 2.9-2.78 | w |
| 32.81-34.22 | 2.73-2.62 | w |
| 35.63-36.99 | 2.52-2.43 | w |
| 41.03-42.86 | 2.2-2.11 | w |
| 44.18-45.83 | 2.05-1.98 | w |
| 44.87-46.57 | 2.02-1.95 | w |
| 46.07-47.35 | 1.97-1.92 | w |
| 48.97-50.42 | 1.86-1.81 | w |

and is thermally stable up to a temperature of greater than 400° C. in one embodiment and 600° C. in another embodiment.

Upon modification by one or more techniques described in U.S. Pat. No. 6,779,975, UZM-35HS if formed and is described by the empirical formula on an anhydrous basis of:

M1ₐⁿ⁺Al₍₁₋ₓ₎EₓSi_{y′}O_{z″}

where M1 is at least one exchangeable cation selected from the group consisting of alkali, alkaline earth metals, rare earth metals, ammonium ion, hydrogen ion and mixtures thereof, “a” is the mole ratio of M1 to (Al+E) and varies from about 0.05 to about 50, “n” is the weighted average valence of M1 and has a value of about +1 to about +3, E is an element selected from the group consisting of gallium, iron, boron, and mixtures thereof, “x” is the mole fraction of E and varies from 0 to 1.0, y′ is the mole ratio of Si to (Al+E) and varies from greater than about 4 to virtually pure silica and z″ is the mole ratio of O to (Al+E) and has a value determined by the equation:

z″=(a·n+3+4·y′)/2

and is characterized in that it has the x-ray diffraction pattern having at least the d-spacings and intensities set forth in Table A above.

The process for preparing the crystalline microporous zeolite described above comprises forming a reaction mixture containing reactive sources of M, R, Al, Si and optionally E and heating the reaction mixture at a temperature of about 150° C. to about 200° C., or about 165° C. to about 185° C., for a time sufficient to form the zeolite, the reaction mixture having a composition expressed in terms of mole ratios of the oxides of:

aM₂O:bR_{2/p}O:1−cAl₂O₃:cE₂O₃:dSiO₂:eH₂O

where “a” has a value of about 0.05 to about 1.25, “b” has a value of about 1.5 to about 40, “c” has a value of 0 to about 1.0, “d” has a value of about 4 to about 40, “e” has a value of about 25 to about 4000. UZM-35 is formed. The UZM-35 is modified by any of the techniques of U.S. Pat. No. 6,776,975 to form the UZM-35HS.

Yet another embodiment of the invention is a hydrocarbon conversion process using the above-described zeolite. The process comprises contacting the hydrocarbon with the zeolite at conversion conditions to give a converted hydrocarbon.

### DETAILED DESCRIPTION OF THE INVENTION

Applicants have prepared an aluminosilicate zeolite whose topological structure is related to MSE as described in Atlas of Zeolite Framework Types, which is maintained by the International Zeolite Association Structure Commission at http://topaz.ethz.ch/IZA-SC/StdAtlas.htm, which has been designated UZM-35. As will be shown in detail, UZM-35 is different from MCM-68 in a number of its characteristics. The instant microporous crystalline zeolite (UZM-35) has an empirical composition in the as-synthesized form and on an anhydrous basis expressed by the empirical formula:

Mₘⁿ⁺R⁺ᵣAl₁₋ₓEₓSi_{y}O_{z}

where M represents a combination of potassium and sodium exchangeable cations. R is a singly charged organoammonium cation, examples of which include but are not limited to the dimethyldipropylammonium cation (DMDPA⁺), choline [(CH₃)₃N(CH₂)₂OH]⁺, ETMA⁺, DEDMA⁺, trimethylpropylammonium, trimethylbutylammonium, dimethyldiethanolammonium, methyltripropylammonium, TEA⁺, TPA⁺ and mixtures thereof and “r” is the mole ratio of R to (Al+E) and varies from about 0.25 to about 2.0. while “m” is the mole ratio of M to (Al+E) and varies from about 0.05 to about 3. The ratio of silicon to (Al+E) is represented by “y” which varies from about 2 to about 30. E is an element which is tetrahedrally coordinated, is present in the framework and is selected from the group consisting of gallium, iron and boron. The mole fraction of E is represented by “x” and has a value from 0 to about 1.0, while “z” is the mole ratio of O to (Al+E) and is given by the equation:

z=(m·n+r+3+4·y)/2.

Where M is only one metal, then the weighted average valence is the valence of that one metal, i.e. +1 or +2. However, when more than one M metal is present, the total amount of:

Mₘⁿ⁺=Mₘ₁⁽ⁿ¹⁾⁺+Mₘ₂⁽ⁿ²⁾⁺+Mₘ₃⁽ⁿ³⁾⁺+ . . .

and the weighted average valence “n” is given by the equation:

$$n = \frac{{m}_{1} \cdot {n}_{1} + {m}_{2} \cdot {n}_{2} + {m}_{3} \cdot {n}_{3} + …}{{m}_{1} + {m}_{2} + {m}_{3} …}$$

The microporous crystalline zeolite, UZM-35, is prepared by a hydrothermal crystallization of a reaction mixture prepared by combining reactive sources of M, R, aluminum, silicon and optionally E. The sources of aluminum include but are not limited to aluminum alkoxides, precipitated aluminas, aluminum metal, aluminum salts and alumina sols. Specific examples of aluminum alkoxides include, but are not limited to aluminum ortho sec-butoxide and aluminum ortho isopropoxide. Sources of silica include but are not limited to tetraethylorthosilicate, colloidal silica, precipitated silica and alkali silicates. Sources of the E elements include but are not limited to alkali borates, boric acid, precipitated gallium oxyhydroxide, gallium sulfate, ferric sulfate, and ferric chloride. Sources of the M metals, potassium and sodium, include the halide salts, nitrate salts, acetate salts, and hydroxides of the respective alkali metals. R is an organoammonium cation selected from the group consisting of dimethyldipropylammonium, choline, ETMA, DEDMA, TEA, TPA, trimethylpropylammonium, trimethylbutylammonium, dimethyldiethanolammonium and mixtures thereof, and the sources include the hydroxide, chloride, bromide, iodide and fluoride compounds. Specific examples include without limitation dimethyldipropylammonium hydroxide, dimethyldipropylammonium chloride, dimethyldipropylammonium bromide, ethyltrimethylammonium hydroxide, diethyldimethylammonium hydroxide, tetraethylammonium hydroxide, tetrapropylammonium hydroxide, tetrapropylammonium chloride.

The reaction mixture containing reactive sources of the desired components can be described in terms of molar ratios of the oxides by the formula:

aM₂O:bR_{2/p}O:1−cAl₂O₃:cE₂O₃:dSiO₂:eH₂O

where “a” varies from about 0.05 to about 1.25, “b” varies from about 1.5 to about 40, “c” varies from 0 to 1.0, “d” varies from about 4 to about 40, and “e” varies from about 25 to about 4000. If alkoxides are used, it is preferred to include a distillation or evaporative step to remove the alcohol hydrolysis products. The reaction mixture is now reacted at a temperature of about 150° C. to about 200° C., about 165° C. to about 185° C., or about 170° C. to about 180° C., for a period of about 1 day to about 3 weeks and preferably for a time of about 5 days to about 12 days in a sealed reaction vessel under autogenous pressure. After crystallization is complete, the solid product is isolated from the heterogeneous mixture by means such as filtration or centrifugation, and then washed with deionized water and dried in air at ambient temperature up to about 100° C. It should be pointed out that UZM-35 seeds can optionally be added to the reaction mixture in order to accelerate the formation of the zeolite.

A preferred synthetic approach to make UZM-35 utilizes the charge density mismatch concept, which is disclosed in U.S. Pat. No. 7,578,993 and Studies in Surface Science and Catalysis, (2004), Vol. 154A, 364-372. The method disclosed in U.S. Pat. No. 7,578,993 employs quaternary ammonium hydroxides to solubilize aluminosilicate species, while crystallization inducing agents such as alkali and alkaline earth metals and more highly charged organoammonium cations are often introduced in a separate step. Once some UZM-35 seeds have been generated using this approach, the seeds can be used in a single step synthesis of UZM-35, using, for example, a combination of dimethyldipropylammonium hydroxide and the alkali cations. The use of commercially available dimethyldipropylammonium hydroxide to prepare UZM-35 offers a great economic advantage over the structure directing agents previously employed (N,N,N′,N′-tetraalkylbicyclo[2.2.2.]oct-7-ene-2,3:5,6-dipyrrolidinium dication, and N,N,N′,N′-tetraalkylbicyclo[2.2.2.]octane-2,3:5,6-dipyrrolidinium dication) to prepare aluminosilicates with the MSE topology. Additionally, dimethyldipropyl ammonium hydroxide can be employed as the hydroxide or the chloride in concert with other inexpensive organoammonium hydroxides using the charge density mismatch concept to reduce costs even further.

The UZM-35 aluminosilicate zeolite, which is obtained from the above-described process, is characterized by the x-ray diffraction pattern, having at least the d-spacings and relative intensities set forth in Table A below.

TABLE A

| 2θ | d (Å) | I/Io % |
|---|---|---|
| 6.45-6.8 | 13.7-13 | m |
| 6.75-7.13 | 13.1-12.4 | m-vs |
| 7.86-8.26 | 11.25-10.7 | m |
| 8.64-9.04 | 10.23-9.78 | m |
| 9.51-10.09 | 9.3-8.77 | m-vs |
| 10.62-11.23 | 8.33-7.88 | w-m |
| 13.4-14.22 | 6.61-6.23 | w-m |
| 14.76-15.55 | 6-5.7 | w |
| 17.63-18.37 | 5.03-4.83 | w |
| 19.17-19.91 | 4.63-4.46 | w-m |
| 19.64-20.56 | 4.52-4.32 | m |
| 20.18-21.05 | 4.4-4.22 | w-m |
| 20.7-21.57 | 4.29-4.12 | w-m |
| 21.36-22.28 | 4.16-3.99 | vs |
| 22.17-23.6 | 4.01-3.77 | m-s |
| 24.12-25.23 | 3.69-3.53 | w |
| 25.6-26.94 | 3.48-3.31 | m |
| 26.37-27.79 | 3.38-3.21 | m |
| 27.02-28.42 | 3.3-3.14 | m |
| 27.53-28.89 | 3.24-3.09 | m |
| 28.7-30.09 | 3.11-2.97 | m |
| 29.18-30.72 | 3.06-2.91 | w-m |
| 30.19-31.73 | 2.96-2.82 | m |
| 30.83-32.2 | 2.9-2.78 | w |
| 32.81-34.22 | 2.73-2.62 | w |
| 35.63-36.99 | 2.52-2.43 | w |
| 41.03-42.86 | 2.2-2.11 | w |
| 44.18-45.83 | 2.05-1.98 | w |
| 44.87-46.57 | 2.02-1.95 | w |
| 46.07-47.35 | 1.97-1.92 | w |
| 48.97-50.42 | 1.86-1.81 | w |

As will be shown in detail in the examples, the UZM-35 material is thermally stable up to a temperature of at least 400° C. and in another embodiment, up to about 600° C.

As synthesized, the UZM-35 material will contain some of the exchangeable or charge balancing cations in its pores. These exchangeable cations can be exchanged for other cations, or in the case of organic cations, they can be removed by heating under controlled conditions. Because UZM-35 is a large pore zeolite, it is also possible to remove some organic cations directly by ion exchange. The UZM-35 zeolite may be modified in many ways to tailor it for use in a particular application. Modifications include calcination, ion-exchange, steaming, various acid extractions, ammonium hexafluorosilicate treatment, or any combination thereof, as outlined for the case of UZM-4M in U.S. Pat. No. 6,776,975 B1 which is incorporated by reference in its entirety. Properties that are modified include porosity, adsorption, Si/A1 ratio, acidity, thermal stability, etc.

The UZM-35 compositions which are modified by one or more techniques described in the '975 patent (herein UZM-35HS) are described by the empirical formula on an anhydrous basis of:

M1ⁿ⁺Al₍₁₋ₓ₎EₓSi_{y′}O_{z″}

where M1 is at least one exchangeable cation selected from the group consisting of alkali, alkaline earth metals, rare earth metals, ammonium ion, hydrogen ion and mixtures thereof, “a” is the mole ratio of M1 to (Al+E) and varies from about 0.05 to about 50, “n” is the weighted average valence of M1 and has a value of about +1 to about +3, E is an element selected from the group consisting of gallium, iron, boron, and mixtures thereof, “x” is the mole fraction of E and varies from 0 to 1.0, y′ is the mole ratio of Si to (Al+E) and varies from greater than about 4 to virtually pure silica and z″ is the mole ratio of O to (Al+E) and has a value determined by the equation:

z″=(a·n+3+4y′)/2

The resulting UZM-35HS has the same x-ray diffraction pattern as shown for UZM-35 in Table A shown above. In other words, The UZM-35HS aluminosilicate zeolite, which is obtained from the process described herein, is characterized by the x-ray diffraction pattern, having at least the d-spacings and relative intensities set forth in Table A above,

UZM-35HS is obtained by treating a starting zeolite having the topology of UZM-35 with, for example, a fluorosilicate solution or slurry; calcination or steaming followed by acid extraction or ion-exchange; acid extraction or any combination of these treatments in any order.

The acid treatment involves contacting a UZM-35 starting material with an acid in order to remove some of the aluminum from the framework resulting in the UZM-35HS zeolite. Although it is known that aluminum can be extracted from the framework by acids, it is not predictable whether the resulting product will retain a substantial portion of its crystallinity or whether the structure will collapse resulting in an amorphous material. The acids which can be used in carrying out acid extraction include, but are not limited to, mineral acids, carboxylic acids and mixtures thereof. Examples of these include sulfuric acid, nitric acid, hydrochloric acid, ethylene diaminetetraacetic acid (EDTA), citric acid, oxalic acid, and the like. The concentration of the acid which can be used is not critical to the success, but it is conveniently between about 1 wt. % to about 80 wt. % acid and preferably between 5 wt. % and 40% wt % acid. Acid extraction conditions typically include a temperature of about 10° C. to 100° C. for a time of about 10 minutes to about 24 hrs. After treatment with the acid, the resulting UZM-35HS zeolite is isolated by means of filtration, washed with deionized water, and dried at ambient temperature up to about 100° C. The extent of dealumination achieved by the acid extraction depends on the cation form of the starting UZM-35 zeolite as well as the acid concentration and the time and temperature over which the extraction is conducted. For example, if organic cations are present in the starting UZM-35 zeolite, the extent of dealumination will be slight compared to a UZM-5 zeolite in which organic cations have been removed. Convenient ways of removing organic cations include calcination, ammonia calcination, steaming and ion exchange. Calcination conditions usually include temperatures of about 300° C. to about 600° C. for a time of about 2 to about 24 hours, steaming conditions include a temperature of about 400° C. to about 850° C. with from about 1% steam to about 100% steam for a time of about 10 minutes to about 48 hours and preferably a temperature of about 500° C. to about 600° C., steam concentration of about 5 to about 50% and a time of about 1 to about 2 hours. Ammonium ion exchange conditions are namely a temperature of about 15° C. to about 100° C. and a time of about 20 minutes to about 50 hours. Ion exchange can be carried out with a solution comprising a cation selected from the group consisting of alkali metals, alkali earth metals, rare earth metals, hydrogen ion, ammonium ion, and mixtures thereof. By carrying out this ion exchange this ion exchange, the cation is exchanged for a secondary or different cation. In a preferred embodiment, the UZM-35HS zeolite composition after the steaming or calcining steps is contacted with an ion exchange solution comprising an ammonium salt. Examples of ammonium salts include but are not limited to ammonium nitrate, ammonium chloride, ammonium bromide, and ammonium acetate. The ammonium ion containing solution can optionally contain mineral acid such as but not limited to nitric acid, hydrochloric, sulfuric and mixtures thereof. The concentration of the mineral acid is that amount necessary to give a ratio of H⁺ to NH4⁺ of 0 to 1. This ammonium exchange aids in removing any debris present in the pores after steaming and or calcination treatments. After having undergone any of the dealumination treatments as described above, the UZM-35HS is usually dried.

By virtually pure silica is meant that virtually all the aluminum and/or the E metals have been removed from the framework. It is well know that it is virtually impossible to remove all the aluminum and/or E metal. Numerically, a zeolite is virtually pure silica when y′ has a value of at least 3,000, preferably 10,000 and most preferably 20,000. Thus, ranges for y′ are from 4 to 3,000 preferably greater than 10 to about 3,000; 4 to 10,000 preferably greater than 10 to about 10,000 and 4 to 20,000 preferably greater than 10 to about 20,000.

In specifying the proportions of the zeolite starting material or adsorption properties of the zeolite product and the like herein, the “anhydrous state” of the zeolite will be intended unless otherwise stated. The term “anhydrous state” is employed herein to refer to a zeolite substantially devoid of both physically adsorbed and chemically adsorbed water.

The crystalline UZM-35 and UZM-35HS zeolites can be used for separating mixtures of molecular species, removing contaminants through ion exchange and catalyzing various hydrocarbon conversion processes. Separation of molecular species can be based either on the molecular size (kinetic diameter) or on the degree of polarity of the molecular species.

The UZM-35 and UZM-35HS zeolites can also be used as a catalyst or catalyst support in various hydrocarbon conversion processes. Hydrocarbon conversion processes are well known in the art and include cracking, hydrocracking, alkylation of both aromatics and isoparaffin, isomerization of paraffin and poly-alkylbenzenes such as xylene, trans-alkylation of poly-alkybenzene with benzene or mono-alkybenzenes, disproportionation of mono-alkybenzenes, polymerization, reforming, hydrogenation, dehydrogenation, transalkylation, dealkylation, hydration, dehydration, hydrotreating, hydrodenitrogenation, hydrodesulfurization, methanation and syngas shift process. Specific reaction conditions and the types of feeds which can be used in these processes are set forth in U.S. Pat. No. 4,310,440 and U.S. Pat. No. 4,440,871 which are hereby incorporated by reference. Preferred hydrocarbon conversion processes are those in which hydrogen is a component such as hydrotreating or hydrofining, hydrogenation, hydrocracking, hydrodenitrogenation, hydrodesulfurization, etc.

Hydrocracking conditions typically include a temperature in the range of about 204° C. to about 649° C. (400° to 1200° F.) or about 316° C. to about 510° C. (600° F. and 950° F.). Reaction pressures are in the range of atmospheric to about 24,132 kPa g (3,500 psig), or between about 1379 to about 20,685 kPa g (200 to 3000 psig). Contact times usually correspond to liquid hourly space velocities (LHSV) in the range of about 0.1 hr⁻¹ to 15 hr⁻¹, preferably between about 0.2 and 3 hr⁻¹. Hydrogen circulation rates are in the range of 178 to about 8,888 std. m³/m³ (1,000 to 50,000 standard cubic feet (scf) per barrel of charge), or about 355 to about 5,333 std. m³/m³ (about 2,000 to about 30,000 scf per barrel of charge). Suitable hydrotreating conditions are generally within the broad ranges of hydrocracking conditions set out above.

The reaction zone effluent is normally removed from the catalyst bed, subjected to partial condensation and vapor-liquid separation and then fractionated to recover the various components thereof. The hydrogen, and if desired some or all of the unconverted heavier materials, are recycled to the reactor. Alternatively, a two-stage flow may be employed with the unconverted material being passed into a second reactor. Catalysts of the subject invention may be used in just one stage of such a process or may be used in both reactor stages.

Catalytic cracking processes are preferably carried out with the UZM-35 composition using feedstocks such as gas oils, heavy naphthas, deasphalted crude oil residua, etc. with gasoline being the principal desired product. Temperature conditions of about 454° C. to about 593° C. (about 850° F. to about 1100° F.), LHSV values of 0.5 to 10 and pressure conditions of from about 0 to about 344 kPa g (about 0 to 50 psig) are suitable.

Alkylation of aromatics usually involves reacting an aromatic (C₂ to C₁₂), especially benzene, with a monoolefin to produce a linear alkyl substituted aromatic. The process is carried out at an aromatic: olefin (e.g., benzene:olefin) ratio of between 1:1 and 30:1, a olefin LHSV of about 0.3 to about 10 hr⁻¹, a temperature of about 100° to about 250° C. and pressures of about 1379 kPa g to about 6895 kPa g (about 200 to about 1000 psig). Further details on apparatus may be found in U.S. Pat. No. 4,870,222 which is incorporated by reference.

Alkylation of isoparaffins with olefins to produce alkylates suitable as motor fuel components is carried out at temperatures of −30° to 40° C., pressures from about atmospheric to about 6,895 kPa (1,000 psig) and a weight hourly space velocity (WHSV) of 0.1 to about 120. Details on paraffin alkylation may be found in U.S. Pat. No. 5,157,196 and U.S. Pat. No. 5,157,197, which are incorporated by reference.

The following examples are presented in illustration of this invention and are not intended as undue limitations on the generally broad scope of the invention as set out in the appended claims.

The structure of the UZM-35 and UZM-35HS zeolites was determined by x-ray analysis. The x-ray patterns presented in the following examples were obtained using standard x-ray powder diffraction techniques. The radiation source was a high-intensity, x-ray tube operated at 45 kV and 35 ma. The diffraction pattern from the copper K-alpha radiation was obtained by appropriate computer based techniques. Flat compressed powder samples were continuously scanned at 2° to 56° (20). Interplanar spacings (d) in Angstrom units were obtained from the position of the diffraction peaks expressed as 0 where 0 is the Bragg angle as observed from digitized data. Intensities were determined from the integrated area of diffraction peaks after subtracting background, “Iₒ” being the intensity of the strongest line or peak, and “I” being the intensity of each of the other peaks.

As will be understood by those skilled in the art the determination of the parameter 2θ is subject to both human and mechanical error, which in combination can impose an uncertainty of about ±0.4° on each reported value of 2θ. This uncertainty is, of course, also manifested in the reported values of the d-spacings, which are calculated from the 2θ values. This imprecision is general throughout the art and is not sufficient to preclude the differentiation of the present crystalline materials from each other and from the compositions of the prior art. In some of the x-ray patterns reported, the relative intensities of the d-spacings are indicated by the notations vs, s, m, and w which represent very strong, strong, medium, and weak, respectively. In terms of 100×I/Iₒ, the above designations are defined as:

w=0-15; m=15-60: s=60-80 and vs=80-100

In certain instances the purity of a synthesized product may be assessed with reference to its x-ray powder diffraction pattern. Thus, for example, if a sample is stated to be pure, it is intended only that the x-ray pattern of the sample is free of lines attributable to crystalline impurities, not that there are no amorphous materials present.

In order to more fully illustrate the invention, the following examples are set forth. It is to be understood that the examples are only by way of illustration and are not intended as an undue limitation on the broad scope of the invention as set forth in the appended claims.

### Example 1

An aluminosilicate solution was prepared by first mixing 16.64 aluminum hydroxide (27.78% Al) and 526.79 g dimethyldipropylammonium hydroxide, 18.8% solution, with vigorous stirring. After thorough mixing, 252.98 g of Ludox™ AS-40 (40% SiO₂) was added. The reaction mixture was homogenized for an additional hour with a high speed mechanical stirrer and placed in an oven at 100° C. overnight. Analysis showed the resulting aluminosilicate solution contained 6.52 wt. % Si and 0.64 wt. % Al yielding a Si/Al ratio of 9.78.

To a 150 g portion of the aluminosilicate solution prepared in Example 1, a composite aqueous NaOH/KOH solution containing 1.44 g of NaOH (98%) and 2.02 g of KOH dissolved in 20.0 g distilled water was added with vigorous stirring and the reaction mixture was homogenized for an additional 30 minutes. A 24 g portion of the reaction mixture was transferred to a 45 ml Parr stainless steel autoclave which was heated to 175° C. and maintained at that temperature for 120 hrs. The solid product was recovered by centrifugation, washed with de-ionized water, and dried at 100° C.

The solid products were recovered by centrifugation, washed with de-ionized water and dried at 95° C. The product was identified as UZM-35 by xrd. Representative diffraction lines observed for the product are shown in Table 1. The product composition was determined by elemental analysis to consist of the following mole ratios: Si/Al=7.92, Na/Al=0.1, K/Al=0.48.

TABLE 1

| 2θ | d (Å) | I/I₀ % |
|---|---|---|
| 6.65 | 13.26 | m |
| 6.95 | 12.69 | m |
| 8.10 | 10.90 | m |
| 8.87 | 9.95 | m |
| 9.76 | 9.05 | m |
| 10.83 | 8.13 | w |
| 13.76 | 6.43 | w |
| 15.22 | 5.81 | w |
| 18.00 | 4.92 | w |
| 19.46 | 4.55 | m |
| 19.62 | 4.52 | m |
| 20.06 | 4.42 | m |
| 20.63 | 4.3 | m |
| 21.1 | 4.20 | m |
| 21.76 | 4.08 | vs |
| 21.92 | 4.05 | m |
| 22.07 | 4.03 | m |
| 22.55 | 3.93 | m |
| 22.73 | 3.90 | m |
| 23.08 | 3.85 | s |
| 23.42 | 3.79 | m |
| 23.51 | 3.77 | m |
| 24.04 | 3.69 | m |
| 24.53 | 3.62 | w |
| 25.9 | 3.43 | m |
| 25.99 | 3.42 | w |
| 26.27 | 3.38 | m |
| 26.92 | 3.3 | m |
| 27.57 | 3.23 | m |
| 27.76 | 3.21 | m |
| 28.17 | 3.16 | m |
| 28.86 | 3.09 | w |
| 29.27 | 3.04 | m |
| 29.72 | 3.00 | w |
| 30.26 | 2.95 | w |
| 30.91 | 2.88 | m |
| 31.38 | 2.84 | w |
| 33.61 | 2.68 | w |
| 34.65 | 2.58 | w |
| 35.43 | 2.53 | w |
| 36.18 | 2.48 | w |
| 41.77 | 2.16 | w |
| 44.7 | 2.02 | w |
| 45.32 | 1.99 | w |
| 45.63 | 1.98 | w |
| 46.55 | 1.94 | w |
| 47.62 | 1.90 | w |
| 47.94 | 1.89 | w |
| 49.70 | 1.83 | w |
| 51.06 | 1.78 | w |

Scanning Electron Microscopy (SEM) revealed crystals of square shaped morphology, approximately 100 by 350 nm in size. This sample was calcined at 540° C. for 10 hrs under nitrogen and then air. Representative diffraction lines observed for the product are shown in Table 2.

TABLE 2

| 2θ | d (Å) | I/I₀ % |
|---|---|---|
| 6.72 | 13.13 | m |
| 7.02 | 12.57 | vs |
| 8.0 | 11.04 | m |
| 8.2 | 10.77 | m |
| 8.3 | 10.64 | m |
| 8.98 | 9.83 | m |
| 9.87 | 8.94 | vs |
| 11.00 | 8.03 | m |
| 11.29 | 7.82 | w |
| 13.85 | 6.38 | m |
| 14.17 | 6.24 | w |
| 14.95 | 5.91 | w |
| 15.04 | 5.88 | w |
| 17.72 | 4.99 | w |
| 17.90 | 4.95 | w |
| 19.56 | 4.53 | m |
| 19.64 | 4.51 | m |
| 19.70 | 4.50 | m |
| 20.16 | 4.40 | m |
| 20.64 | 4.29 | w |
| 21.15 | 4.19 | w |
| 21.86 | 4.06 | vs |
| 21.98 | 4.04 | s |
| 22.07 | 4.02 | m |
| 22.62 | 3.92 | m |
| 22.72 | 3.91 | s |
| 23.27 | 3.91 | vs |
| 24.08 | 3.69 | m |
| 24.69 | 3.60 | w |
| 25.29 | 3.51 | w |
| 26.28 | 3.38 | m |
| 27.12 | 3.28 | m |
| 27.66 | 3.22 | m |
| 28.28 | 3.15 | m |
| 28.98 | 3.07 | w |
| 29.36 | 3.03 | m |
| 29.99 | 2.97 | w |
| 30.38 | 2.93 | m |
| 31.02 | 2.88 | m |
| 31.54 | 2.83 | w |
| 33.46 | 2.67 | w |
| 34.68 | 2.58 | w |
| 35.07 | 2.55 | w |
| 35.84 | 2.50 | w |
| 36.29 | 2.47 | w |
| 39.37 | 2.28 | w |
| 41.92 | 2.15 | w |
| 44.96 | 2.01 | w |
| 45.72 | 1.98 | w |
| 46.74 | 1.94 | w |
| 47.82 | 1.9 | w |
| 48.13 | 1.88 | w |
| 49.75 | 1.83 | W |

### Example 2

An aluminosilicate reaction solution was prepared by first mixing 37.17 g of aluminum hydroxide (27.78% Al) and 1053.58 g of dimethyldipropylammonium hydroxide (18.8% solution), while stirring vigorously. After thorough mixing, 505.96 g Ludox™ AS-40 (SiO₂, 40%) was added. The reaction mixture was homogenized for 1 hour with a high speed mechanical stirrer, sealed in a Teflon bottle and placed in an oven overnight at 100° C. Analysis showed the aluminosilicate solution contained 6.16 wt. % Si and 0.67 wt. % Al (Si/Al=8.83).

A 100.0 g portion of the above aluminosilicate solution was continuously stirred. A composite aqueous solution containing 2.38 g of KOH and 0.3 g of NaOH dissolve in 15 g H₂O was added, dropwise, to the aluminosilicate solution. After the addition was completed, the resulting reaction mixture was homogenized for 1 hour, transferred to (4) 45 ml Parr stainless steel autoclave which was heated to 175° C. and maintained at that temperature for 216 hrs. The solid product was recovered by centrifugation, washed with de-ionized water, and dried at 100° C.

The solid product from each of these samples was recovered by centrifugation, washed with de-ionized water and dried at 95° C. The products resulting from all four reactions were identified by xrd to be UZM-35. Table 3 shows representative diffraction lines observed for the sample that was reacted for 9 days. Elemental analysis gave a product composition in mole ratios of: Si/Al=7.58, Na/A1=0.033, K/Al=0.63, C/N=6, N/Al=0.43.

TABLE 3

| 2θ | d (Å) | I/I₀ % |
|---|---|---|
| 6.56 | 13.46 | m |
| 6.84 | 12.91 | s |
| 8.10 | 10.90 | m |
| 8.80 | 10.03 | m |
| 9.69 | 9.11 | m |
| 10.80 | 8.18 | w |
| 13.69 | 6.45 | w |
| 14.17 | 6.01 | w |
| 15.10 | 5.86 | w |
| 15.88 | 5.57 | w |
| 18.01 | 4.91 | w |
| 19.48 | 4.55 | w |
| 19.98 | 4.44 | m |
| 20.52 | 4.32 | w |
| 21.00 | 4.22 | m |
| 21.68 | 4.09 | vs |
| 22.49 | 3.94 | m |
| 23.04 | 3.85 | s |
| 24.31 | 3.65 | m |
| 24.61 | 3.61 | w |
| 25.85 | 3.44 | m |
| 26.14 | 3.40 | m |
| 26.85 | 3.31 | m |
| 27.68 | 3.22 | m |
| 28.15 | 3.16 | m |
| 29.20 | 3.05 | m |
| 29.90 | 2.98 | m |
| 30.82 | 2.89 | m |
| 31.33 | 2.85 | w |
| 32.49 | 2.75 | w |
| 33.28 | 2.68 | w |
| 34.42 | 2.60 | w |
| 34.84 | 2.57 | w |
| 35.32 | 2.53 | w |
| 35.69 | 2.51 | w |
| 36.10 | 2.48 | w |
| 37.59 | 2.39 | w |
| 41.75 | 2.16 | w |
| 44.67 | 2.02 | w |
| 45.11 | 2.00 | w |
| 45.45 | 1.99 | w |
| 46.10 | 1.96 | w |
| 46.50 | 1.95 | w |
| 47.01 | 1.93 | w |
| 47.62 | 1.90 | w |
| 49.7 | 1.83 | w |

### Example 3

An aluminosilicate reaction solution was prepared by first mixing 37.17 g of aluminum hydroxide (27.78% Al) and 1053.58 g of dimethyldipropylammonium hydroxide (18.8% solution), while stirring vigorously. After thorough mixing, 505.96 g Ludox™ AS-40 (SiO₂, 40%) was added. The reaction mixture was homogenized for 1 hour with a high speed mechanical stirrer, sealed in a Teflon bottle and placed in an oven overnight at 100° C. Analysis showed the aluminosilicate solution contained 6.16 wt. % Si and 0.67 wt. % Al (Si/Al=8.83).

A 1200 g portion of the above aluminosilicate solution was continuously stirred. A composite aqueous solution containing 28.56 g of KOH and 3.6 g of NaOH dissolve in 150 g H₂O, was added, dropwise, to the aluminosilicate solution. After the addition was completed, the resulting reaction mixture was homogenized for 1 hour, transferred to a 2000 ml Parr stainless steel autoclave which was heated to 175° C. and maintained at that temperature for 216 hrs. The solid product was recovered by centrifugation, washed with de-ionized water, and dried at 100° C.

The solid product from each of these samples was recovered by centrifugation, washed with de-ionized water and dried at 95° C. The products resulting from this reaction were identified by xrd to be UZM-35. Elemental analysis gave a product composition in mole ratios of: Si/Al=7.57, Na/A1=0.028, K/Al=0.73, N/Al=0.37. This sample was calcined at 540° C. for 10 hrs under nitrogen and then air. Representative diffraction lines observed for the product are shown in Table 4.

TABLE 4

| 2θ | d (Å) | I/I₀ % |
|---|---|---|
| 6.54 | 13.5 | m |
| 6.85 | 12.88 | m |
| 8.10 | 10.90 | m |
| 8.82 | 10.01 | m |
| 9.67 | 9.13 | m |
| 10.80 | 8.18 | m |
| 11.08 | 7.97 | w |
| 13.67 | 6.46 | m |
| 14.84 | 5.96 | w |
| 15.21 | 5.81 | w |
| 15.61 | 5.67 | w |
| 15.91 | 5.56 | w |
| 17.47 | 5.07 | w |
| 17.87 | 4.95 | w |
| 19.52 | 4.54 | m |
| 19.96 | 4.44 | m |
| 20.54 | 4.32 | m |
| 21.16 | 4.19 | m |
| 21.67 | 4.09 | vs |
| 21.89 | 4.05 | s |
| 22.54 | 3.94 | s |
| 23.08 | 3.85 | vs |
| 24.45 | 3.63 | m |
| 24.65 | 3.60 | w |
| 25.06 | 3.55 | m |
| 25.84 | 3.44 | m |
| 26.14 | 3.40 | m |
| 26.46 | 3.36 | m |
| 26.90 | 3.31 | m |
| 27.48 | 3.21 | m |
| 27.73 | 3.21 | m |
| 28.19 | 3.16 | m |
| 28.66 | 3.11 | w |
| 29.18 | 3.05 | m |
| 29.58 | 3.01 | w |
| 29.88 | 2.98 | m |
| 30.21 | 2.95 | m |
| 30.80 | 2.90 | m |
| 31.38 | 2.84 | w |
| 33.32 | 2.68 | w |
| 34.52 | 2.59 | w |
| 34.79 | 2.57 | w |
| 35.69 | 2.51 | w |
| 36.15 | 2.48 | w |
| 41.70 | 2.16 | w |
| 44.83 | 2.01 | w |
| 45.46 | 1.99 | w |
| 46.52 | 1.95 | w |
| 47.54 | 1.91 | w |
| 47.88 | 1.89 | w |
| 49.56 | 1.83 | w |

### Example 4

This example describes the modification of a UZM-35 material. A 10 g portion of a UZM-35 sample (Si/Al=7.57) was calcined in a nitrogen atmosphere, ramping at 3° C./min to 540° C. and holding there for an additional hour before changing the atmosphere to air and continuing the calcination for another 9 hr. A solution was prepared by first diluting 2 g of HNO₃ (69%) followed by dissolving 10 g of NH₄NO₃ in 120 g de-ionized water. This solution was heated to 75° C. before adding the calcined UZM-35. The slurry was stirred for 1 hr at 75° C. The product was isolated by filtration, washed with de-ionized water and dried at 100° C. for 12 hrs.

The product was identified as UZM-35HS via x-ray powder diffraction. Elemental analyses confirmed an increase in Si/Al ratio to Si/Al=8.3, Na/Al=0.01, K/Al=0.44.

### Example 5

This example demonstrates the modification of a UZM-35 material. A 20 g portion of a UZM-35 sample (Si/Al=7.57) was calcined under a nitrogen atmosphere by ramping at 3° C./min to 560° C. and holding there for 1 hr before changing the atmosphere to air and continuing the calcination for another 9 hr. Separately, a solution was prepared by dissolving 20 g of NH₄NO₃ in 490 g de-ionized water. The solution was heated to 75° C. before adding the calcined UZM-35. The slurry was stirred for 1 hr at 75° C. The product was isolated by filtration, washed with de-ionized water and dried at 100° C. for 12 hrs.

The product was identified as UZM-35HS via x-ray powder diffraction. Elemental analyses of this sample shows a Si/Al ratio to Si/Al=8.0, Na/Al=0.01, K/Al=0.47.

### Example 6

An aluminosilicate solution was prepared by first mixing 37.17 aluminum hydroxide (27.78% Al) and 1053.58 g dimethyldipropylammonium hydroxide, 18.8% solution, with vigorous stirring. After thorough mixing, 505.96 g of Ludox™ AS-40 (40% SiO₂) was added. The reaction mixture was homogenized for an additional hour with a high speed mechanical stirrer and placed in an oven at 100° C. overnight. Analysis showed the resulting aluminosilicate solution contained 6.16 wt. % Si and 0.67 wt. % Al yielding a Si/Al ratio of 8.83.

To a 100 g portion of the aluminosilicate solution prepared in Example 6 above, an aqueous NaOH solution containing 1.98 g of NaOH (98%) in 10.0 g distilled water was added with vigorous stirring and the reaction mixture was homogenized for an additional 30 minutes. A 24 g portion of the reaction mixture was transferred to a 45 ml Parr stainless steel autoclave which was heated to 175° C. and maintained at that temperature for 144 hrs. The solid product was recovered by centrifugation, washed with de-ionized water, and dried at 100° C.

The solid products were recovered by centrifugation, washed with de-ionized water and dried at 95° C. The product was identified as MOR by xrd.

### Example 7

An aluminosilicate solution was prepared by first mixing 37.17 aluminum hydroxide (27.78% Al) and 1053.58 g dimethyldipropylammonium hydroxide, 18.8% solution, with vigorous stirring. After thorough mixing, 505.96 g of Ludox™ AS-40 (40% SiO₂) was added. The reaction mixture was homogenized for an additional hour with a high speed mechanical stirrer and placed in an oven at 100° C. overnight. Analysis showed the resulting aluminosilicate solution contained 6.16 wt. % Si and 0.67 wt. % Al yielding a Si/Al ratio of 8.83.

To a 150 g portion of the aluminosilicate solution prepared in Example 6, an aqueous KOH solution containing 3.84 g of KOH dissolved in 20.0 g distilled water was added with vigorous stirring and the reaction mixture was homogenized for an additional 30 minutes. A 24 g portion of the reaction mixture was transferred to a 45 ml Parr stainless steel autoclave which was heated to 175° C. and maintained at that temperature for 264 hrs. The solid product was recovered by centrifugation, washed with de-ionized water, and dried at 100° C.

The solid products were recovered by centrifugation, washed with de-ionized water and dried at 95° C. The product was identified as ZSM-5 by xrd.

1. A microporous crystalline zeolite having a three-dimensional framework of at least AlO₂ and SiO₂ tetrahedral units and an empirical composition in the as synthesized and anhydrous basis expressed by an empirical formula of:
M1ₐⁿ⁺Al₍₁₋ₓ₎EₓSi_{y′}O_{z″}
where M1 is at least one exchangeable cation selected from the group consisting of alkali, alkaline earth metals, rare earth metals, ammonium ion, hydrogen ion and mixtures thereof, “a” is the mole ratio of M1 to (Al+E) and varies from about 0.05 to about 50, “n” is the weighted average valence of M1 and has a value of about +1 to about +3, E is an element selected from the group consisting of gallium, iron, boron, and mixtures thereof, “x” is the mole fraction of E and has a value from 0 to 1.0, y′ is the mole ratio of Si to (Al+E) and varies from greater than about 4 to virtually pure silica and z″ is the mole ratio of O to (Al+E) and has a value determined by the equation:
z″=(a·n+3+4·y′)/2
and is characterized in that it has the x-ray diffraction pattern having at least the d-spacings and intensities set forth in Table A:
TABLE A

| 2θ | d (Å) | I/Io % |
|---|---|---|
| 6.45-6.8 | 13.7-13 | m |
| 6.75-7.13 | 13.1-12.4 | m-vs |
| 7.86-8.26 | 11.25-10.7 | m |
| 8.64-9.04 | 10.23-9.78 | m |
| 9.51-10.09 | 9.3-8.77 | m-vs |
| 10.62-11.23 | 8.33-7.88 | w-m |
| 13.4-14.22 | 6.61-6.23 | w-m |
| 14.76-15.55 | 6-5.7 | w |
| 17.63-18.37 | 5.03-4.83 | w |
| 19.17-19.91 | 4.63-4.46 | w-m |
| 19.64-20.56 | 4.52-4.32 | m |
| 20.18-21.05 | 4.4-4.22 | w-m |
| 20.7-21.57 | 4.29-4.12 | w-m |
| 21.36-22.28 | 4.16-3.99 | vs |
| 22.17-23.6 | 4.01-3.77 | m-s |
| 24.12-25.23 | 3.69-3.53 | w |
| 25.6-26.94 | 3.48-3.31 | m |
| 26.37-27.79 | 3.38-3.21 | m |
| 27.02-28.42 | 3.3-3.14 | m |
| 27.53-28.89 | 3.24-3.09 | m |
| 28.7-30.09 | 3.11-2.97 | m |
| 29.18-30.72 | 3.06-2.91 | w-m |
| 30.19-31.73 | 2.96-2.82 | m |
| 30.83-32.2 | 2.9-2.78 | w |
| 32.81-34.22 | 2.73-2.62 | w |
| 35.63-36.99 | 2.52-2.43 | w |
| 41.03-42.86 | 2.2-2.11 | w |
| 44.18-45.83 | 2.05-1.98 | w |
| 44.87-46.57 | 2.02-1.95 | w |
| 46.07-47.35 | 1.97-1.92 | w |
| 48.97-50.42 | 1.86-1.81 | w |

and is thermally stable up to a temperature of at least 400° C.

2. The zeolite of claim 1 where “x” is zero.

3. The zeolite of claim 1 where the zeolite is thermally stable up to a temperature of at least 600° C.

4. A hydrocarbon conversion process comprising contacting a hydrocarbon stream with a catalyst at hydrocarbon conversion conditions to give a converted product, the catalyst comprising a UZM-35HS microporous crystalline zeolite, wherein the UZM-35HS has a three-dimensional framework of at least AlO₂ and SiO₂ tetrahedral units and an empirical composition in the as synthesized and anhydrous basis expressed by an empirical formula of:
M1ₐⁿ⁺Al₍₁₋ₓ₎EₓSi_{y′}O_{z″}
where M1 is at least one exchangeable cation selected from the group consisting of alkali, alkaline earth metals, rare earth metals, ammonium ion, hydrogen ion and mixtures thereof, “a” is the mole ratio of M1 to (Al+E) and varies from about 0.05 to about 50, “n” is the weighted average valence of M1 and has a value of about +1 to about +3, E is an element selected from the group consisting of gallium, iron, boron, and mixtures thereof, “x” is the mole fraction of E and has a value from 0 to 1.0, y′ is the mole ratio of Si to (Al+E) and varies from greater than about 4 to virtually pure silica and z″ is the mole ratio of 0 to (Al+E) and has a value determined by the equation:
z″=(a·n+3+4·y′)/2
and is characterized in that it has the x-ray diffraction pattern having at least the d-spacings and intensities set forth in Table A
TABLE A

| 2θ | d (Å) | I/Io % |
|---|---|---|
| 6.45-6.8 | 13.7-13 | m |
| 6.75-7.13 | 13.1-12.4 | m-vs |
| 7.86-8.26 | 11.25-10.7 | m |
| 8.64-9.04 | 10.23-9.78 | m |
| 9.51-10.09 | 9.3-8.77 | m-vs |
| 10.62-11.23 | 8.33-7.88 | w-m |
| 13.4-14.22 | 6.61-6.23 | w-m |
| 14.76-15.55 | 6-5.7 | w |
| 17.63-18.37 | 5.03-4.83 | w |
| 19.17-19.91 | 4.63-4.46 | w-m |
| 19.64-20.56 | 4.52-4.32 | m |
| 20.18-21.05 | 4.4-4.22 | w-m |
| 20.7-21.57 | 4.29-4.12 | w-m |
| 21.36-22.28 | 4.16-3.99 | vs |
| 22.17-23.6 | 4.01-3.77 | m-s |
| 24.12-25.23 | 3.69-3.53 | w |
| 25.6-26.94 | 3.48-3.31 | m |
| 26.37-27.79 | 3.38-3.21 | m |
| 27.02-28.42 | 3.3-3.14 | m |
| 27.53-28.89 | 3.24-3.09 | m |
| 28.7-30.09 | 3.11-2.97 | m |
| 29.18-30.72 | 3.06-2.91 | w-m |
| 30.19-31.73 | 2.96-2.82 | m |
| 30.83-32.2 | 2.9-2.78 | w |
| 32.81-34.22 | 2.73-2.62 | w |
| 35.63-36.99 | 2.52-2.43 | w |
| 41.03-42.86 | 2.2-2.11 | w |
| 44.18-45.83 | 2.05-1.98 | w |
| 44.87-46.57 | 2.02-1.95 | w |
| 46.07-47.35 | 1.97-1.92 | w |
| 48.97-50.42 | 1.86-1.81 | w |

and is thermally stable up to a temperature of at least 400° C.

5. The process of claim 4 where the hydrocarbon conversion process is selected from the group consisting of alkylation, trans-alkylation, isomerization, olefin dimerization, olefin oligomerization, and dewaxing.
